(19) United States
(12) Patent Application Publication
(10) Pub. No.: US 2024/0158817 A1
(43) Pub. Date: May 16, 2024

(54) METHODS FOR SEPARATING PLANT MATTER INTO ANATOMICAL STRUCTURES

(71) Applicant: Battelle Energy Alliance, LLC, Idaho Falls, ID (US)

(72) Inventors: Bradley D. Wahlen, Rigby, ID (US); Asif Hasan Rony, Idaho Falls, ID (US); William A. Smith, Idaho Falls, ID (US); Lynn M. Wendt, Idaho Falls, ID (US)

(21) Appl. No.: 18/511,399

(22) Filed: Nov. 16, 2023

Related U.S. Application Data
(60) Provisional application No. 63/383,986, filed on Nov. 16, 2022.

(51) Int. Cl. C12P 7/10; C12N 9/24

(52) U.S. Cl. CPC C12P 7/10; C12N 9/2402; C12Y 302/01015

(57) ABSTRACT

Methods of separating plant matter are described herein. The methods may include contacting monocotyledonous plant matter with one or more enzymes or one or more organisms expressing one or more enzymes, where the monocotyledonous plant matter includes vascular bundles, pith, and rind. Following the enzymatic treatment, additional processing of the plant matter may be undertaken through mechanical means. The low-intensity mechanical processing may dislodge the pith, rind, and vascular bundles from one another while minimizing energy consumption and potential damage to the plant matter. Further processing may include sorting mechanisms such as mechanical screening or pneumatic air classification. The incorporation of enzymatic treatment prior to mechanical separation may enhance the efficiency and effectiveness of separating the rind, pith, and vascular bundles from one another compared to conventional mechanical separation methods.

### CROSS-REFERENCE TO RELATED APPLICATION

This application claims the benefit under 35 U.S.C. § 119(e) of U.S. Provisional Patent Application Ser. No. 63/383,986, filed Nov. 16, 2022, the disclosure of which is hereby incorporated herein in its entirety by this reference.

### STATEMENT REGARDING FEDERALLY SPONSORED RESEARCH OR DEVELOPMENT

This invention was made with government support under Contract Number DE-AC07-05-ID14517 awarded by the United States Department of Energy. The government has certain rights in the invention.

### TECHNICAL FIELD

This disclosure relates generally to methods for separating plant material into anatomical structures. More specifically, the disclosure relates to methods for separating monocotyledonous plant matter into vascular bundles, pith, and rind using enzymes to facilitate the separation.

### BACKGROUND

The efficient use of agricultural waste as a resource for fuel and chemical production has gained increasing attention. Although research on the use of corn stalks for insulated building materials in the U.S. goes back to 1930 with the National Bureau of Standards, the heterogeneous nature of these materials creates significant challenges for industrial processing. Monocotyledonous plants (e.g., monocots) have anatomical structures that include the pith, rind, and vascular bundles. The pith, mainly composed of nutrient-rich, large, thin-walled parenchyma cells, functions in storage and structural support inside the plant. The rind serves as a protective layer for the plant against environmental factors and lends mechanical strength, consisting predominantly of collenchyma and sclerenchyma cells. Vascular bundles, which are composed of four concentrically arranged hollow tubes made up of xylem and phloem tissues, play an essential role in transporting water and nutrients inside the plant and have unique properties that make them ideal for diverse applications.

While the pith and rind structures have many polymers in common, the pith and rind differ significantly in texture and density. Furthermore, unlike in dicotyledonous plants where vascular bundles are concentrically arranged, in monocotyledonous plants, the vascular bundles are dispersed throughout the stem. Conventional separation methods, such as mechanical fractionation, are limited in their effectiveness to isolate the vascular bundles.

### BRIEF SUMMARY

In one embodiment, a method of separating plant matter includes contacting monocotyledonous plant matter with one or more enzymes where the monocotyledonous plant matter includes vascular bundles, pith, and rind, storing the one or more enzymes and monocotyledonous plant matter under anaerobic conditions, heating the one or more enzymes and monocotyledonous plant matter during the anaerobic conditions to form treated plant matter, drying the treated plant matter, milling the treated plant matter, and separating the treated plant matter into the vascular bundles, pith, and rind.

In another embodiment, a method of separating plant matter into anatomical structures includes combining monocotyledonous plant matter with one or more organisms expressing one or more of a glycohydrolase or an oxidoreductase where the plant matter includes vascular bundles, pith, and rind, storing the one or more organisms and monocotyledonous plant matter under anaerobic conditions, heating the one or more organisms and monocotyledonous plant matter during the anaerobic conditions to form treated plant matter, drying the treated plant matter, milling the treated plant matter, and separating, based on size, the treated plant matter into vascular bundles, pith, and rind.

In another embodiment, a method for separating anatomical structures of corn stover includes contacting corn stover, where the corn stover includes corn stalks, with one or more pectinases, placing the one or more pectinases and corn stalks under anaerobic conditions, heating the one or more pectinases and corn stalks to about 40° C. during the anaerobic conditions to form enzyme treated corn stalks, drying the enzyme treated corn stalks, milling the enzyme treated corn stalks, and separating the enzyme treated corn stalks into anatomical structures, where the anatomical structures include vascular bundles, pith, and rind.

### BRIEF DESCRIPTION OF THE DRAWINGS

FIG. 1 is a flow diagram showing a process for separating plant matter into anatomical structures in accordance with embodiments of the disclosure;

FIG. 2 shows anatomical structures of plant matter;

FIGS. 3A-3D show isolated corn stalk anatomical structures in accordance with embodiments of the disclosure;

FIGS. 4A and 4B show corn stalk with and without enzyme treatment in accordance with embodiments of the disclosure;

FIGS. 5A-5C show corn stalk at 50× magnification after low, high, and no enzyme treatment in accordance with embodiments of the disclosure;

FIG. 6 is a graph of rind thickness following low, high, and no enzyme treatment in accordance with embodiments of the disclosure;

FIGS. 7A-7C depict axial cross sections of a vascular bundle isolated from corn stalk in accordance with embodiments of the disclosure; and

FIG. 8 is a μ-CT scan of a vascular bundle isolated from corn stalk in accordance with embodiments of the disclosure.

### DETAILED DESCRIPTION

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings and does not connote or depend on any specific preference or order, except where the context clearly indicates otherwise.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

According to embodiments described herein, methods for the separation of monocotyledonous (e.g., monocot) plant matter into anatomical structures may include contacting the plant matter with one or more enzymes, storing the one or more enzymes and plant matter under anaerobic conditions, heating the enzyme-treated plant matter during the anaerobic condition, and maintaining the anaerobic condition to form treated plant matter. Following the enzymatic treatment, the treated plant matter may be mechanically processed and screened to separate and isolate the anatomical structures. The enzyme may target and degrade polysaccharide linkages interspersed among the anatomical structures of the treated plant matter, improving the effectiveness of separation compared to conventional mechanical separation methods. The methods according to embodiments of the disclosure may, for example, be used to separate and isolate pith, rind, and vascular bundles present in the monocotyledonous plant matter. Compared to conventional methods of plant matter separation, the methods according to embodiments of the disclosure increase the purity of the isolated anatomical structures, thus boosting the economic viability of downstream feedstock conversion processes.

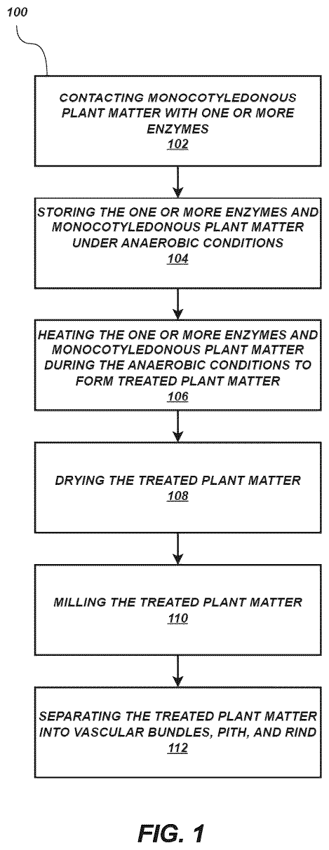

FIG. 1 is a flow diagram showing a process 100 according to embodiments of the disclosure for separating plant matter into vascular bundles, pith, and rind. The process 100 includes the act 102 of contacting monocotyledonous plant matter with one or more enzymes; the act 104 of storing the one or more enzymes and monocotyledonous plant matter under anaerobic conditions; the act 106 of heating the one or more enzymes and monocotyledonous plant matter during the anaerobic conditions to form treated plant matter; the act 108 of drying the treated plant matter; the act 110 of milling the treated plant matter; and the act 112 of separating the treated plant matter into vascular bundles, pith, and rind.

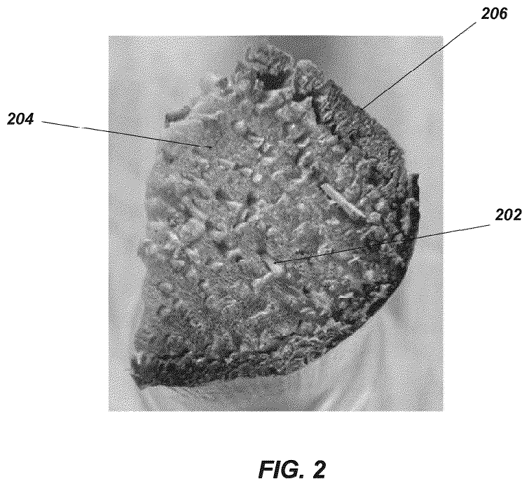

The plant matter may be a monocotyledonous plant matter, including, but not limited to, corn (e.g., maize), energy cane, Miscanthus, switchgrass, cool season grasses such as orchard grass, bromegrass, and Timothy, and warm season grasses such as fescue, Bermuda grass, and ryegrass, rice straw, wheat straw, barley straw, oat straw, and sorghum. Monocotyledonous plants exhibit distinct anatomical structures, as depicted in FIG. 2. Specifically, the monocotyledonous plants include the vascular bundles 202—complex arrangements of xylem and phloem tissues—embedded in a ground tissue known as pith 204. The pith 204 functions as an undifferentiated matrix that provides structural support and storage for the monocot plants. Surrounding this inner matrix of pith 204 and vascular bundles 202 is a dense, fibrous outer layer referred to as a rind 206. The rind 206 functions as a protective layer, contributing to the plant's overall resilience against environmental stressors such as pests and climate variations. Compared to dicotyledonous plants, which have a layered structure with distinct cortex and vascular bundles organized in a ring, the monocotyledonous plant matter lacks this level of tissue differentiation and specialization. The heterogeneity of the monocotyledonous plant matter makes the anatomical structures in the monocotyledonous plants difficult to isolate from one another.

The monocotyledonous plant matter may constitute a feedstock for the process 100 and may be supplied as a bale or in another form. The monocotyledonous plant matter may, for example, be an agricultural waste material, such as corn stover, which includes a combination of cobs, leaves, husks, and stalks of the corn. However, other monocotyledonous plant matter may be used as the feedstock, such as energy cane, miscanthus, or sorghum. The monocotyledonous plant matter may be pre-processed so that only a portion that contains vascular bundles 202 is used in the process 100. In other words, pre-processing may be conducted to separate the feedstock into a portion containing a higher relative amount of the vascular bundles 202 compared to other portions of the feedstock. The process 100, according to embodiments of the disclosure, may be used to separate outer portions, such as the rind 206, of the monocotyledonous plant matter from inner portions, such as the pith 204. The process 100, according to embodiments of the disclosure, may be used to isolate the vascular bundles 202 from the other components (e.g., the anatomical structures) of the monocotyledonous plant matter without substantially damaging the vascular bundles 202. The isolated vascular bundles 202 may, therefore, be recovered substantially intact.

The act 102 of contacting monocotyledonous plant matter with one or more enzymes may include adding one or more glycohydrolase enzymes, oxidoreductase enzymes, or one or more organisms expressing the one or more glycohydrolase enzymes or oxidoreductase enzymes to the plant matter. The enzyme may be selected to react with polysaccharide linkages in the plant matter, causing breakdown of the rind and the pith. The glycohydrolase(s) may include, but are not limited to, a pectinase such as a polygalacturonase, pectin lyase, pectin transeliminase, or pectin methyl esterase; a hemicellulase such as a xylanase or a mannanase; a cellulase such as an endoglucanase, exoglucanase, cellobiohydrolase, or β-glucosidase; an amylase such as an α-amylase or a β-amylase; or an oxidoreductase such as a ligninase. The specific enzyme or combination of enzymes may be tailored according to the plant matter's composition and the desired anatomical structures to be isolated. By way of example only, a combination of enzymes (e.g., an enzyme cocktail including pectin transeliminase, pectin methyl esterase, and polygalacturonase) may be used for plant matter with a high pectin content, whereas for plant matter rich in lignocellulosic material, the combination of enzymes may include ligninase, endoglucanase, and cellobiohydrolase. Each enzyme of the combination of enzymes may be selected to target a different chemical bond in the polysaccharide linkages of the respective polymers, accelerating their breakdown.

The enzyme(s) may be combined with the plant matter at a concentration of from about 1 mg enzyme/g biomass (dry basis) to about 5 mg enzyme/g biomass (dry basis), such as from about 1 mg enzyme/g biomass to about 2 mg enzyme/g biomass or from about 2 mg enzyme/g biomass to about 5 mg enzyme/g biomass. The plant matter may also be inoculated with one or more organisms expressing the enzyme(s). The organism(s) may be bioengineered to express the enzyme(s). The organism(s) may include, for example, an anaerobic microbe. The enzyme(s) or organism(s) may be introduced into the plant matter by, for example, spraying or mixing. Utilizing organisms rather than purified enzymes may reduce the overall cost of the feedstock conversion process.

The feedstock containing the monocotyledonous plant matter may be produced, harvested, and collected by conventional techniques. The feedstock may initially include the rind 206 tightly adhered to the pith 204. The feedstock may be preprocessed into the portion containing the higher relative amount of the vascular bundles 202. Following production, harvest, and collection, but prior to transportation, the plant matter may be heated and stored for a time period that ranges from about 1 week to about 4 weeks. The enzyme(s) or organism(s) expressing the enzyme(s) may be introduced to the plant matter before or during the storage. The enzyme(s) or organism(s) may be, for example, sprayed into and/or combined with the plant matter. The moisture content of the plant matter may be suitable to facilitate enzymatic functionality and may be from about 20 wt. % to about 90 wt. % (as-received basis, also known as wet or total weight basis), such as from about 20 wt. % to about 30 wt. %, from about 30 wt. % to about 40 wt. %, from about 40 wt. % to about 50 wt. %, from about 50 wt. % to about 60 wt. %, from about 60 wt. % to about 70 wt. %, from about 70 wt. % to about 80 wt. %, or from about 80 wt. % to about 90 wt. %. The storage environment, e.g., a storage tank, silage pile, or another type of containment, may have a temperature ranging from about 20° C. to about 60° C., such as from about 20° C. to about 30° C., from about 30° C. to about 40° C., from about 40° C. to about 50° C., or from about 50° C. to about 60° C.

The enzymatically treated plant matter may be stored in a suitable storage vessel that is configured to provide a desired storage environment. The storage environment may be an anaerobic environment. The anaerobic environment may inhibit the growth of aerobic microbes that may compete with the enzymatic process or bioengineered organisms. The anaerobic environment may also minimize the risk of oxidation reactions that could adversely affect the quality of the feedstock or efficacy of the enzymatic treatment.

The duration of storage may be related to factors such as a temperature at which the process 100 is conducted and enzyme concentration. Extended storage periods may permit the utilization of lower temperatures and enzyme concentrations during the process 100. By way of example only, if the plant matter is stored for about 2 weeks, an enzyme concentration of about 5 mg enzyme/g biomass (dry basis) may be used; if the plant matter is stored for about 4 weeks, a lower enzyme concentration of about 1 mg enzyme/g biomass (dry basis) may be used, as the enzymes will have more time to break down the target polysaccharides.

The treated plant matter may be dried. The act 108 of drying the treated plant matter may include heating the treated plant matter to a temperature of from about 40° C. to about 100° C.

Once the treated plant matter is dried, the act 110 of milling the treated plant matter may be conducted to separate the dried and treated plant matter while maintaining the structural integrity of the anatomical structures. The milling may include using milling equipment such as, but not limited to, a hammer mill, a roller mill, a compression mill, or an attrition mill to break up the treated plant matter. The milling machine may be operated at a low intensity to reduce operational costs and maintain the structural integrity of the anatomical structures of the plant matter, namely the rind 206, the pith 204, and the vascular bundles 202, while effecting their separation from each other. The dimensions and parameters of the milling equipment may be adjusted depending on various factors, including but not limited to, the scale of the operation, the type of plant matter being processed, and the specific components being targeted for separation. For example, the size of the milling chamber, the rotational speed of the milling components, and the throughput rate may be adjusted depending on the particular plant matter and the objectives of the milling operation.

The act 112 of performing size separation on the milled and treated plant matter may include separating the anatomical structures based on parameters such as size, morphology, and/or density to enable a more targeted use of the anatomical structures once separated. For example, the vascular bundles 202 may be less dense than the pith 204, and the pith 204 may be less dense than the rind 206. Additionally, the vascular bundles 202 exhibit an elongated and slender shape, whereas the pith 204 and rind 206 are relatively cuboid. By employing a mechanical screening technique to separate the anatomical structures, the pith 204 may be separated from the rind 206 and the vascular bundles 202 based on their different sizes. By way of example only, stacked sieves may be used, in which sieves with varying mesh sizes are arranged sequentially to separate the anatomical structures based on size. Similarly, an air classification technique such as pneumatic air classification, in which a stream of air is used to separate the anatomical structures based on their aerodynamic properties, may separate the pith 204 from the rind 206 and the vascular bundles 202, based on their different densities and aspect ratios. The pith 204, rind 206, and vascular bundles 202 may also be physically separated using a gravity separator. Following the enzymatic treatment and the mechanical fractionation, one of the separated portions includes the vascular bundles 202, while other of the separated portions include the rind 206 delaminated from the pith 204. The separated pith 204, rind 206, and vascular bundles 202 may be recovered as separate fractions following the size separation. The recovered vascular bundles 202 may be substantially pure, such as being substantially free of the pith. By way of example only, the vascular bundles 202 prepared by the methods according to embodiments of the disclosure may be greater than about 80% pure, such as greater than about 85% pure, greater than about 90% pure, or greater than about 95% pure. The recovered vascular bundles 202 may exhibit a length of from about 2 cm to about 10 cm and a diameter of from about 90 μm to about 200 μm.

The methods detailed in embodiments of the disclosure may be used in small-scale operations but also are highly adaptable for implementation in extensive large-scale operations. In such contexts, the plant matter may be stored in specifically designed silage piles, capable of containing up to about 50,000 tons of plant matter, facilitating large-scale enzymatic treatment. Specialized equipment, such as stacking machines, reclaimers, or front-end loaders, may be employed to efficiently manage the plant matter in these silage piles.

For the mechanical fractionation and screening, industrial-scale equipment may be used to achieve significant throughput. Specifically, the mechanical processing may handle up to about 50 tons of plant matter per hour, depending on the equipment and the specific requirements of the material being processed. Likewise, screening processes may be designed to operate at scales ranging up to about 50 tons per hour, depending on the particular anatomical structures being isolated and the desired purity levels.

The methods disclosed herein offer increased separation efficiency compared to conventional systems of mechanical separation and allow for the unexpected separation of intact vascular bundles 202, rind 206, and pith 204 from whole plant matter. The effective separation of the vascular bundles 202, rind 206, and pith 204 improves the efficiency and economic viability of downstream feedstock conversion processes. For example, compared to the cob, leaves, and husks of corn, corn stalks have by far the highest sugar potential in terms of glucan content, yet only 50% of that glucan is fermentable in a conventional treatment. Therefore, with conventional treatment, additional preprocessing of the corn stalks is conducted for reduced yields. Tissue level fractionation of corn stalks has the potential to significantly increase the fermentable sugar potential. The scalability of the methods according to embodiments of the disclosure allows for seamless integration into existing agricultural and industrial infrastructures, making them a viable solution for large-scale applications that demand high throughput and efficiency.

While conventional methods may categorize biomass into broad types like cobs, leaves, husks, and stalks, they fail to adequately isolate the closely adhered pith, rind, and vascular bundles in monocots. This restricts processing efficiency and limits the potential for developing specialized products that leverage the unique characteristics of the different anatomical structures. By separating and recovering the monocot plant matter into the pith 204, rind 206, and vascular bundles 202, the recovered anatomical structures may have more targeted use. The separation of rind 206 and vascular bundles 202 from pith 204, according to embodiments of the disclosure, will facilitate processing of agricultural waste material in biorefineries and provide co-products to help drive down the cost of biofuels. With a thermal conductivity value of 0.039 W/(m·K), the pith 204 may be used in applications requiring insulative properties, liquid absorbency, vibration, and impact shock absorbing properties. The rind 206 may be used in applications that utilize its long strong fibers for making paper and building materials. Vascular bundles 202 have utility in applications such as microfluidics or as natural scaffolding for the creation of other products that take advantage of their unique shape and internal structure. The vascular bundles 202 may also be used in applications where their capillary properties, selectivity to solutes, and microporosity are beneficial, such as in production of renewable graphite, water purification, energy storage, and pollution abatement.

### Examples

Using an air classifier, corn stover bale was classified into cob, stalks, leaves and husk. The corn stover stalks were brought to about 55% moisture content and were subjected to one of three conditions: treatment with a pectinase at a low concentration of 1 mg enzyme/g dry corn stalk; treatment with a pectinase at a high concentration of 5 mg enzyme/g dry corn stalk; or a control condition with no enzyme treatment. The pectinase cocktail was purchased from Sigma-Aldrich® (PN P2611) and included a pectin transeliminase, a polygalacturonase, and a pectinesterase derived from Aspergillus aculeatus.

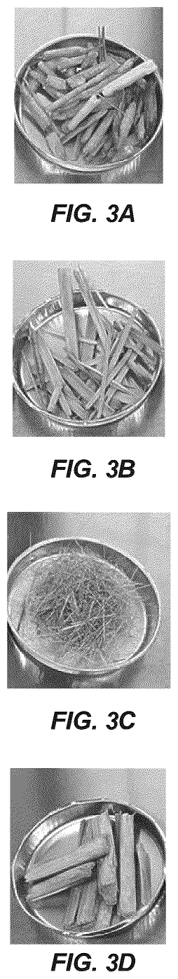

In all three conditions, the corn stalks were stored at 40° C. for two weeks in anaerobic storage, a length that represents a queuing pile at a refinery gate. Throughout the storage period, pectinase activity was confirmed by detecting galacturonic acid, a byproduct of pectinase activity. After two weeks, the corn stalks were processed in a hammer mill at 10% power to encourage separation of the pith from the vascular bundles and rind without damaging the components. Following hammer milling, the corn stalk was separated based on size using stacked 2 mm and 6.35 mm sieves. The corn stalk was separated into pith, rind, or vascular bundles. FIG. 3A shows the isolated pith; FIG. 3B shows the isolated rind; FIG. 3C shows the isolated vascular bundles; and FIG. 3D shows the corn stalk before treatment. The high (0.125 g enzyme/g dry corn stalk) enzyme treatment resulted in 45% of available pith being recovered without rind attached, compared to just 28% for the control treatment, representing a 61% increase in separation, as shown in Table 1:

TABLE 1

| Sample | Pith Yield* |
|---|---|
| No Enzyme (avg.) | 28.5% ± 1% |
| Low Enzyme (avg.) | 38.0% ± 4% |
| High Enzyme (avg.) | 45.1% ± 8% |

*g large pith particle liberated/g total pith present in corn stalk

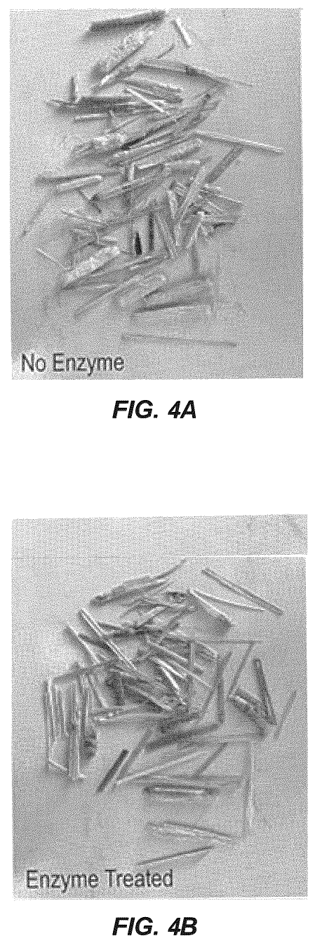

FIGS. 4A and 4B show the effect of pectinase treatment on pith and rind separation. FIG. 4A shows control samples without enzyme treatment, which had a greater number of rind sections with pith attached than the enzyme treated sample, shown in FIG. 4B.

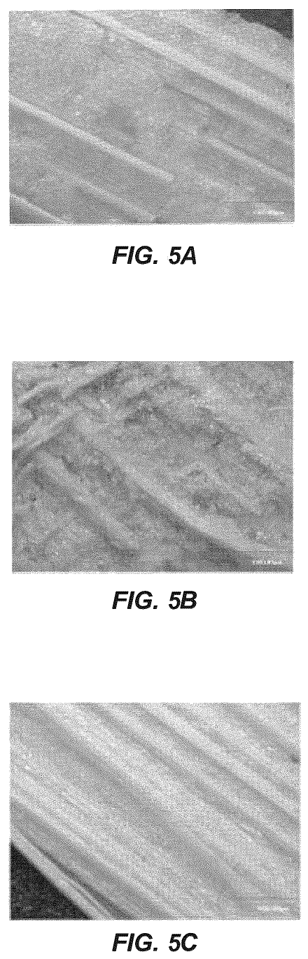

FIGS. 5A-5C show the corn stalk at 50× magnification after being subjected to the control, low enzyme, and high enzyme conditions. FIG. 5A shows the corn stalks after no enzyme treatment; FIG. 5B shows the corn stalk after low (0.025 g enzyme/g dry corn stalk) enzyme treatment; and FIG. 5C shows the corn stalk after high (0.125 g enzyme/g dry corn stalk) enzyme treatment. The enzyme treatment enhanced pith erosion around the vascular bundles in both the low and the high enzyme treatments compared to the no enzyme condition. The effect was seen both in internodes, where the vascular bundles are parallel to one another, and near the nodes, where a more random fiber orientation exists. The amount of pith erosion around the vascular bundles was greater in the high enzyme treatment condition than the low enzyme treatment.

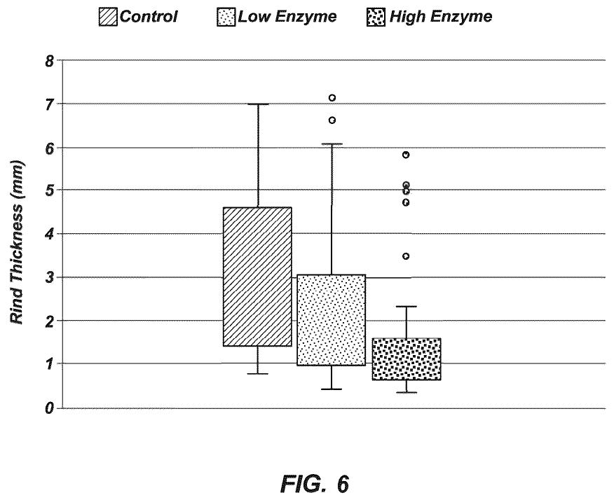

FIG. 6 is a box-plot diagram of the rind thickness of the different conditions following enzyme treatment. The median thickness for nontreated corn stalk was about three times higher than the median thickness of the cornstalk treated with a high enzyme concentration and about two times higher than the cornstalk treated with a low enzyme concentration. The dry matter loss was less than 5%.

The isolated vascular bundles were characterized by micro-computer assisted tomography (R-CT). For X-ray CT of corn stalk vascular bundles, radiograph acquisition was performed by utilizing the ZEISS™ Xradia 520 Versa X-ray microscope (Carl Zeiss X-ray Microscopy Inc., Dublin, CA, USA) at Idaho National Lab's Materials and Fuels Complex. For this experiment, the tungsten X-ray source was operated at 30 kVp accelerating voltage with a target current of 66.13 μA. A rotation stage between the source and the detector enabled the sample rotation between radiographs. Projections were acquired at every 0.14 degrees over 360 degrees of rotation with a total exposure time of 8 seconds. To achieve a high spatial resolution a 4X objective lens with a 2k×2k charge coupled detector (CCD) detector was utilized with the source to rotation axis distance at 12.78 mm and detector to rotation axis distance at 12.65 mm resulting in 1.71 μm isotropic voxel size.

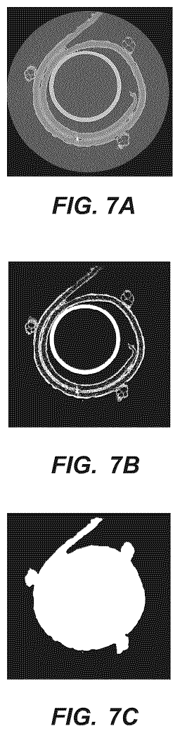

FIGS. 7A-7C show X-ray CT scans of the isolated corn stalk vascular bundles. To reduce the artifacts introduced from movement of the sample during acquisition, the corn stover vascular bundles were taped on to a carbon tube. FIG. 7A shows an axial cross-section image of three corn stalk bundles taped to a carbon tube. Phase contrast was used to increase the complexity of the segmentation. For segmenting the corn stalk vascular bundles, a simple thresholding was utilized followed by a morphological closing operation using a 11×11×11 kernel and morphological filling of isolated values of zeros within the segmented regions. This was equivalent to removing any closed porosity observed within the volume. After applying morphological filters, watershed transform was applied to segment the vascular bundles. FIG. 7B shows an axial cross-section image of the vascular bundle after thresholding; and FIG. 7C shows the sample after morphological filtering.

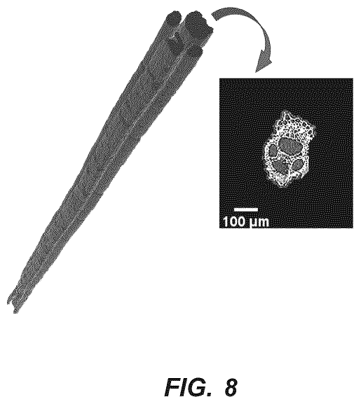

FIG. 8 shows an image reconstructed using ii-CT scan data of a vascular bundle separated from corn stalk. The vascular bundle was found to be a continuous hollow cylindrical structure. The inset image on the right shows a top-down view of the 3D cylindrical structure. The image was reconstructed using several 3-D image analysis techniques. A morphological closing operation with a 7-voxel sphere of radius structuring element was used to identify and digitally reconstruct connected solid spaces such as those surrounding the four large tube features shown in FIG. 8. This was followed by a connected component algorithm to identify and reconstruct the different pore spaces or voids in the corn stalk vascular bundle.

The variation in the measured vascular bundle pore diameters was measured across different vascular bundles. The equivalent diameter was estimated for each slice, which was assumed to represent a cross-section along the long axis of the vascular bundle. The equivalent diameter was determined by first calculating the area of the region of interest, followed by estimating the diameter of a circle which encompasses the same area. A comparison of the mean diameter from different vascular bundles highlights the variation across them. One particular measurement was consistent across vascular bundles, while another showed a large spread in diameters. Two other measurements had similar mean diameters in a given range. The mean diameters of the vascular bundle pores ranged from about 61.6 μm to about 121.0 μm.

Variation in the vascular bundle pore diameters was measured for different vascular bundles. Circularity provides insight into how closely the segmented space resembles a circle. Circularity was estimated by considering the ratio of the perimeter of the segmented region to the equivalent circle using a specified formula. In a continuous binary image, a perfect circle has a circularity of 1, while 0 represents a highly irregular shape. However, small objects or discontinuity can lead to unexpected results. Measurements were taken along the length of various vascular bundles to determine the relative circularity of each measurement. Among the vascular bundles, certain measurements were found to be more circular than others. The circularity of the pores varied from about 0.6 to about 0.9.

The methods according to embodiments of the disclosure utilize long-term storage or queuing to perform enzymatic treatments on monocot plant matter that enhance anatomical fractionation during preprocessing and reduce feedstock variability. Storage pre-treatment may enhance material quality and improve downstream operations, making the production of biofuels and other products from agricultural waste more economically viable.

What is claimed is:

1. A method of separating plant matter comprising:
contacting monocotyledonous plant matter with one or more enzymes, the monocotyledonous plant matter comprising vascular bundles, pith, and rind;
storing the one or more enzymes and monocotyledonous plant matter under anaerobic conditions;
heating the one or more enzymes and monocotyledonous plant matter during the anaerobic conditions to form treated plant matter;
drying the treated plant matter;
milling the treated plant matter; and
separating the treated plant matter into the vascular bundles, pith, and rind.

2. The method of claim 1, wherein contacting monocotyledonous plant matter with one or more enzymes comprises contacting corn stalk with the one or more enzymes.

3. The method of claim 1, wherein contacting monocotyledonous plant matter with one or more enzymes comprises contacting the monocotyledonous plant matter with a pectinase.

4. The method of claim 3, wherein contacting the monocotyledonous plant matter with a pectinase comprises contacting the monocotyledonous plant matter with a pectinase derived from Aspergillus aculeatus.

5. The method of claim 1, wherein contacting monocotyledonous plant matter with one or more enzymes comprises combining the monocotyledonous plant matter and a concentration of from about 1 mg of enzyme/g of monocotyledonous plant matter to about 5 mg of enzyme/g of monocotyledonous plant matter.

6. The method of claim 1, wherein heating the one or more enzymes and the monocotyledonous plant matter during the anaerobic conditions comprises heating the monocotyledonous plant matter to a temperature of from about 40° C. to about 60° C. during the anaerobic conditions.

7. The method of claim 1, wherein heating the one or more enzymes and monocotyledonous plant matter during the anaerobic conditions comprises maintaining the anaerobic conditions for from about 1 week to about 4 weeks.

8. A method of separating plant matter into anatomical structures comprising:
combining monocotyledonous plant matter comprising vascular bundles, pith, and rind with one or more organisms expressing one or more of a glycohydrolase or an oxidoreductase;
storing the one or more organisms and monocotyledonous plant matter under anaerobic conditions;
heating the one or more organisms and monocotyledonous plant matter during the anaerobic conditions to form treated plant matter;
drying the treated plant matter;
milling the treated plant matter; and
separating, based on size, the treated plant matter into vascular bundles, pith, and rind.

9. The method of claim 8, wherein combining monocotyledonous plant matter comprising vascular bundles, pith, and rind with one or more organisms comprises contacting the monocotyledonous plant matter with one or more organisms expressing a pectinase, a hemicellulase, a cellulase, an amylase, an oxidoreductase, or a combination thereof.

10. The method of claim 8, wherein combining monocotyledonous plant matter with one or more organisms expressing one or more of a glycohydrolase or an oxidoreductase comprises combining the monocotyledonous plant matter with one or more organisms expressing a pectinase derived from Aspergillus aculeatus.

11. The method of claim 8, wherein combining monocotyledonous plant matter with one or more organisms expressing one or more of a glycohydrolase or an oxidoreductase comprises delaminating the vascular bundles from the pith.

12. The method of claim 8, wherein combining monocotyledonous plant matter with one or more organisms expressing one or more of a glycohydrolase or an oxidoreductase comprises reacting polysaccharide linkages in the monocotyledonous plant matter with the one or more of the glycohydrolase or the oxidoreductase expressed by the one or more organisms.

13. A method for separating anatomical structures of corn stover comprising:
contacting corn stover comprising corn stalks with one or more pectinases;
placing the one or more pectinases and corn stalks under anaerobic conditions;
heating the one or more pectinases and corn stalks to about 40° C. during the anaerobic conditions to form enzyme treated corn stalks;
drying the enzyme treated corn stalks;
milling the enzyme treated corn stalks; and
separating the enzyme treated corn stalks into anatomical structures comprising vascular bundles, pith, and rind.

14. The method of claim 13, further comprising separating the corn stover into portions, one of the portions comprising the corn stalks and other of the portions comprising cobs, leaves, or husks.

15. The method of claim 13, wherein drying the enzyme treated corn stalks comprises heating the enzyme treated corn stalks to a temperature of from about 40° C. to about 100° C.

16. The method of claim 13, wherein separating the enzyme treated corn stalks into anatomical structures comprising vascular bundles, pith, and rind comprises separating, based on size, the enzyme treated corn stalks into the vascular bundles, pith, and rind.

17. The method of claim 13, further comprising recovering intact vascular bundles.

18. The method of claim 17, wherein recovering intact vascular bundles comprises recovering the vascular bundles exhibiting a length of from about 2 cm to about 10 cm and a diameter of from about 90 μm to about 200 μm.
